(19) United States
(12) Patent Application Publication
(10) Pub. No.: US 2022/0315997 A1
(43) Pub. Date: Oct. 6, 2022

(54) ENHANCED LINKED TARGET CAPTURE

(71) Applicant: NCAN Genomics, Inc., Vancouver (CA)

(72) Inventors: Joel Pel, Vancouver (CA); Andrea Marziali, North Vancouver (CA)

(21) Appl. No.: 17/713,690

(22) Filed: Apr. 5, 2022

Related U.S. Application Data
(60) Provisional application No. 63/170,694, filed on Apr. 5, 2021.

(51) Int. Cl. C12Q 1/6827; C12Q 1/6876; C12Q 1/6855

(52) U.S. Cl. CPC C12Q 1/6827; C12Q 1/6876; C12Q 1/6855; C12Q 2600/156

(57) ABSTRACT

The invention generally relates to using linked target capture probes to profile the adaptive immune system of a subject, detect pathogens, perform spatial sequencing, and isolate mutant sequences.

### RELATED APPLICATIONS

The present application claims the benefit of and priority to U.S. provisional application Ser. No. 63/170,694, filed Apr. 5, 2021.

### FIELD OF THE INVENTION

The invention generally relates to capturing, amplifying and sequencing nucleic acids.

### BACKGROUND

Capturing and sequencing target nucleic acids and regions with sufficient sensitivity while avoiding off-target interactions remains extremely important for accurate and cost-effective research and diagnostics. However, balancing sensitivity and specificity can be difficult, and the desired balance may vary depending on the application.

The adaptive immune system plays a critical role in counteracting pathogens. The basis of the adaptive immune system are T and B cells, which employ V(D)J recombination during their development to produce a vast array of T and B cell receptors (T cell receptors for T cells, and antibodies/immunoglobin for B cells). These receptors can adapt to new pathogens to neutralize them, and thus, sequencing the recombination in a single cell or the full repertoire of all recombinations across many cells is of great interest. Sequencing of the adaptive immune system can elucidate immune response and can be used to improve health outcomes, including diagnosing current disease, detecting immune response to a previous disease (e.g. exposure to (SARS-CoV-2), determining vaccination status, informing and aiding in vaccine development, determining disease treatment, detecting novel pathogens, preventing disease, and detecting disease recurrence. However, cost-effective, accurate, and sensitive means of capturing the entire repertoire of recombinations are still lacking.

Another case where sensitivity is important is pathogen detection where broad-spectrum detection can be useful in identifying the presence of a variety of viruses, bacteria, fungi, protozoa, or viroids that may have limited conserved regions and variable regions that make universal detection difficult.

Another developing application of sequencing is spatial sequencing. Spatial sequencing is a broad collection of methods that generally allow for the determination of RNA sequences with respect to a particular cellular or sub-cellular position. These methods can be broad (whole transcriptome) or targeted but are generally limited to RNA sequencing because of its high abundance and relative ease of capture (e.g. polyA tails). Methods are emerging for high resolution spatial DNA sequencing but are limited in their ability to target particular regions.

For Example, Payne et al. in “In situ genome sequencing resolves DNA sequence and structure in intact biological samples” Science, Vol 371, Issue 6532, 2021, doi: 10.1126/science.aay3446, use rolling circle amplification with universal primers to amplify all available DNA sequences. Incorporated herein by reference.

### SUMMARY

Systems and methods of the invention provide linked target capture techniques with programmed sensitivity and specificity applicable to a variety of sequencing applications. In certain embodiments, linked target capture probes may be designed to target a variety of sequences in the variable (V), joining (J), constant (C) region, or diversity (D) gene regions, such that the combination of linked target capture probes can target all possible V and J combinations from T and B cells in a single reaction. Accordingly, systems and methods of the invention can provide a robust profile of the adaptive immune system.

In certain embodiments, systems and methods of the invention can applied to pathogen detection by designing linked target capture probes to target pathogen sequences. Probes can be designed against conserved regions, such as the 16S or 18S genes in bacteria, and the ITS gene in fungi, such that a single or small set of probes can detect a broad range of pathogens. Since the probe used in linked target capture is not required to initiate PCR priming, capture probes can have variable homology to the target sequence. A broad range of pathogens can be detected by designing capture probes targeting variable regions but requiring only a partial match to successfully capture the target.

Linked target capture techniques of the invention can also be used with circular templates. In certain embodiments, linked target capture can be applied to circular templates to target DNA for spatial sequencing. The linked target capture probes can provide increased specificity into rolling circle based spatial DNA analysis.

In certain embodiments, linked target capture techniques of the invention can be applied to mutation-specific enrichment. Target-specific probes can be mutation-specific such that wild-type and off-target sequences will not be captured, amplified, and sequenced. Accordingly, time and costs can be reduced by avoiding the traditional amplification and sequencing all DNA at a target locus and then determining mutations through sequence analysis.

### BRIEF DESCRIPTION OF THE FIGURES

FIG. 1 illustrates exemplary methods of linked target capture for use in sequencing the adaptive immune system.

FIG. 2 illustrates exemplary methods of linked target capture for use in pathogen detection.

FIGS. 3A and 3B illustrate exemplary methods of linked target capture for use with rolling circle amplification.

FIGS. 4A and 4B illustrate exemplary methods of mutant DNA enrichment using standard linked target capture and mutant-specific linked target capture.

FIG. 5 illustrates mutant-specific linked target capture probes.

### DETAILED DESCRIPTION

The invention generally relates to methods for targeted capture and sequencing of DNA. Linked target capture (LTC) techniques are used wherein linked target capture probes including a universal primer and a target-specific probe are employed and reactions occur under conditions that require the target-specific probe to bind in order to permit binding of the universal primer. Universal primer sites can be attached onto the ends of DNA. The target-specific portion of the linked target capture probe can then be designed to be specific to the target of interest, and the targeted DNA can be sequenced. Linked target capture techniques applicable to the present systems and methods of the invention are described in U.S. application Ser. Nos. 16/239,100; 16/467,870, and 17/269,515 as well as PCT Pub. Nos. WO 2020/141464 and WO 2020/251968, the content of each of which is incorporated herein by reference.

Linked target capture techniques can be used to sequence the immune system, including sequencing of regions formed by V(D)J recombination such as what occurs in the development of T and B cells in the adaptive immune system.

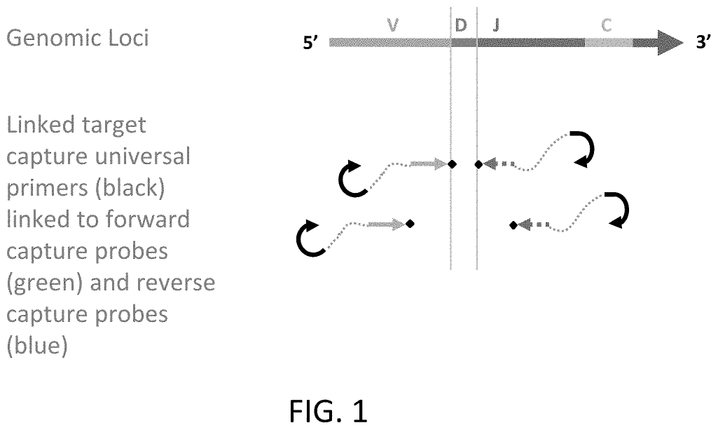

Linked target capture can be used to sequence the adaptive immune system, using DNA, RNA or cDNA as input. Linked target capture probes can be designed in such a way as to determine the immune repertoire. For example in FIG. 1, forward capture probes can be designed against all variable (V) gene regions, reverse capture probes can be designed against all joining (J) gene regions, such that the combination of linked target capture probes can target all possible V and J combinations from T and B cells in a single reaction. Designing reverse capture probes against the V region and forward capture probes against the J region is also possible.

Linked target capture probes can be designed for V and J genes. More than one capture probe can be designed in the same orientation for each V and J region, which may increase recovery efficiency. For example, one, two, three or four capture probes can be designed for each V and/or J region. Probes in these regions may overlap each other by 0, 5, 10, 15 or more bases.

Linked target capture probes can also be designed against any other desired region, such as the constant (C) region or the diversity (D) region.

Sequencing of the linked target capture libraries enables the determination of the adaptive immune sequences, including any sequence, such as the D sequence, between V and J sequences.

Attachment of universal priming sites can be achieved using known methods, such as PCR, ligation, template switching, or transposase.

Linked target capture techniques can be used to detect pathogens, by using capture probes targeting pathogen sequences. Linked target capture followed by sequencing can be used to determine pathogen sequences, including pathogen variants. Pathogens may include viruses, bacteria, fungi, protozoa, or viroids.

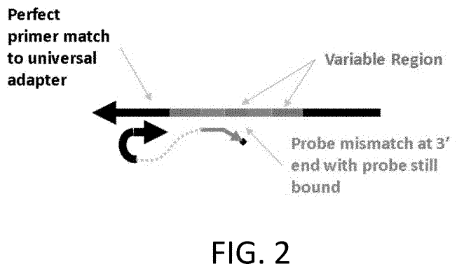

Linked target capture probes can be designed against pathogen sequences. Probes can be designed against conserved regions, such as the 16S or 18S genes in bacteria, and the ITS gene in fungi, such that a single or small set of probes can detect a broad range of pathogens. Since the probe used in linked target capture is not required to initiate PCR priming, capture probes can have variable homology to the target sequence such as in FIG. 2. For example, capture probes can be designed in variable regions where an imperfect match to a probe will still result in capture.

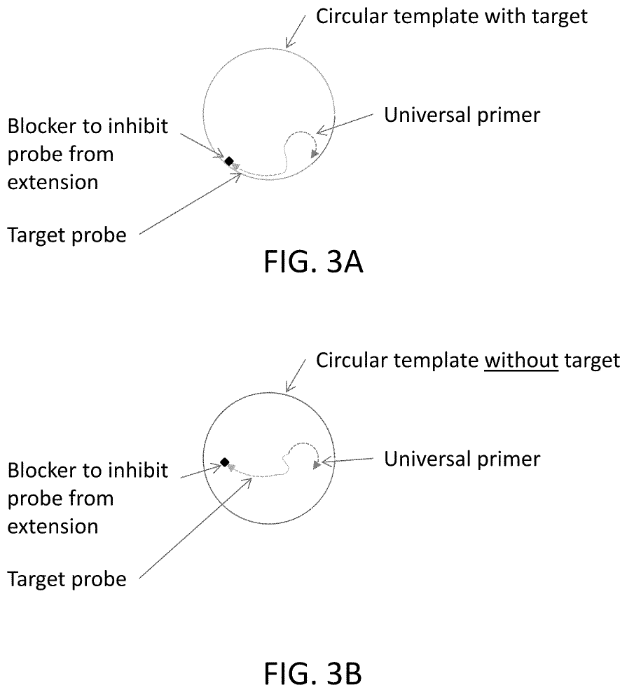

Linked target capture techniques can be used to target DNA for spatial sequencing. For example, linked target capture can be designed to work with circular templates (and then applied to spatial sequencing as described in Payne, 2021), so that only circular templates of interest are targeted in rolling circle amplification, as illustrated in FIG. 3. Accordingly, increased specificity can be incorporated into the rolling circle based spatial DNA analysis. A universal primer can be designed against a universal priming site present in all circular templates. When linked to target probes, universal primers will only provide amplification if the target sequence is present in the circular template (FIG. 3A), but not if the target sequences is not present (FIG. 3B). In this way, only circular DNA templates with the desired targets will be amplified for spatial sequencing.

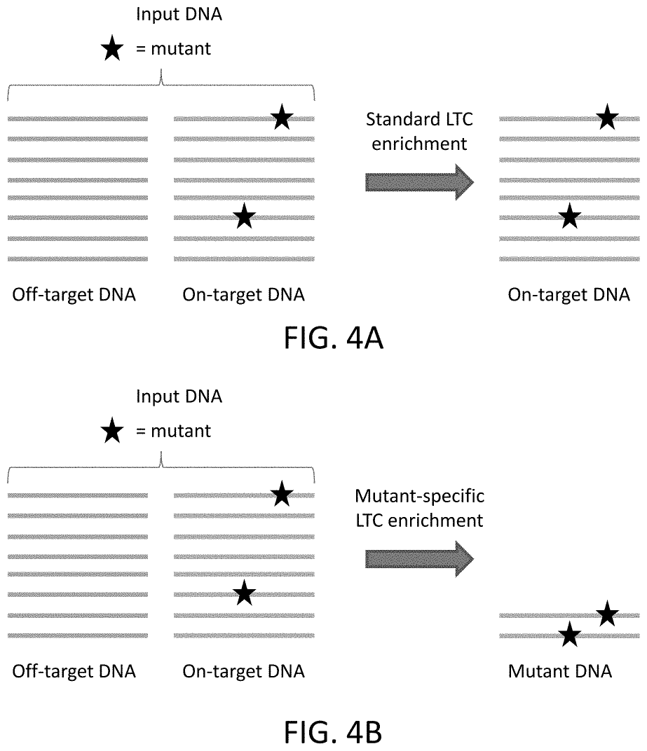

Linked target capture techniques can be used for mutation enrichment as shown in FIG. 4. In certain applications it is desirable to capture only a mutant or a particular allele sequence, such as when detecting minimal residual disease from a known tumour sequence. Mutations from an excised tumour can be used to track the presence of any disease recurrence, such as described by Gydush et al. in “MAESTRO affords ‘breadth and depth’ for mutation testing”, bioRxiv, Jan. 24, 2021, doi: https://doi.org/10.1101/2021.01.22.427323, incorporated herein by reference. By targeting only particular mutants or alleles, abundant wild-type DNA is rejected for sequencing, reducing assay cost significantly.

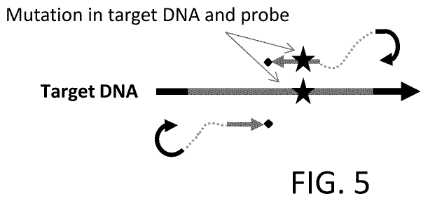

Linked target capture probes can be designed to target only particular mutants or alleles, by making the probes a perfect match to the desired target sequence (FIG. 5). Many mutations may be targeted simultaneously in the same reaction. Additionally, probe modifications may be made to increase specificity for a given mutant or allele. For example, Locked Nucleic Acids (LNAs) may be used at a mutant or other position to increase specificity for a mutant. By designing mutant-specific probes, linked target capture can be utilized to enrich for mutant DNA only, rejecting both off target and wild-type DNA and dramatically reducing sequencing cost (FIG. 4).

Linked target capture probes can include modifications to improve their performance. For example, LNAs can be used to target specific mutants, or increase the melting temperature for a given probe. Intentional mismatches may also be introduced into probes, to reduce the melting temperature of a given sequence, or to reduce the capture rate of undesired sequences. Universal bases may be included, for example to minimize the impact of a possible mutation at a particular position in the target sequence.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

What is claimed is:

1. A method for profiling adaptive immune systems, the method comprising:
attaching universal priming sites to a plurality of nucleic acid fragments from one or more lymphocytes;
exposing the nucleic acid fragments to a plurality of linked capture probes comprising a target probe having affinity to at least a portion of a variable (V), joining (J), constant (C) region, or diversity (D) gene region of a T cell receptor chain, an immunoglobulin heavy chain, or an immunoglobulin light chain, the target probe linked to a universal primer, wherein the exposing step occurs under conditions that require binding of the target probe to the target region to permit binding of the universal primer to the universal priming site;
extending the universal primer to produce a copy of a captured portion of the T cell receptor chain, the immunoglobulin heavy chain, or the immunoglobulin light chain; and
sequencing the copy to profile a subject's adaptive immune system.

2. The method of claim 1, wherein the plurality of linked capture probes comprise capture probes targeting all V gene regions.

3. The method of claim 2, wherein the plurality of linked capture probes further comprises capture probes targeting all J gene regions.

4. The method of claim 1, wherein the plurality of linked capture probes comprise capture probes targeting all C gene regions.

5. The method of claim 1, wherein the plurality of linked capture probes comprise capture probes targeting all D gene regions.

6. The method of claim 1, wherein the plurality of linked capture probes comprise capture probes targeting a plurality of subregions within one or more of the V, J, C, or D gene regions.

7. The method of claim 6, wherein each of the plurality of subregions overlaps with another of the plurality of subregions.

8. The method of claim 7, wherein each of the plurality of subregions overlaps with another of the plurality of subregions by 5 or more bases.

9. The method of claim 1, wherein the one or more lymphocytes are T cells.

10. The method of claim 1, wherein the one or more lymphocytes are B cells.

11. The method of claim 1, wherein the one or more lymphocytes comprise both B cells and T cells.

12. The method of claim 1, further comprising detecting the subject's exposure to a pathogen based on the subject's adaptive immune system profile.

13. The method of claim 1, further comprising determining the subject's vaccination status based on the subject's adaptive immune system profile.

14. The method of claim 1, wherein the attaching step comprises ligation, PCR amplification, template switching, or transposition with a transposase.

15. The method of claim 1, wherein the nucleic acid fragments comprise one or more of DNA, RNA, or cDNA.

16. A method for pathogen detection, the method comprising:
attaching universal priming sites to a plurality of nucleic acid fragments obtained from a sample comprising one or more pathogens;
exposing the nucleic acid fragments to a plurality of linked capture probes comprising a target probe having affinity to at least a portion of a plurality of pathogen sequences, the target probe linked to a universal primer, wherein the exposing step occurs under conditions that require binding of the target probe to the target pathogen sequence to permit binding of the universal primer to the universal priming site;
extending the universal primer to produce a copy of a captured portion of the captured variable region; and
sequencing the copy to identify the one or more pathogens.

17. The method of claim 16, wherein the target probe has variable homology to a variable region of a plurality of pathogens.

18. The method of claim 16, wherein the target probe has affinity to a conserved region selected from the group consisting of 16S, 18S, and ITS genes.

19. The method of claim 16, wherein the attaching step comprises ligation, PCR amplification, template switching, or transposition with a transposase.

20. A method for targeted capture of nucleic acids, the method comprising:
exposing a circular nucleic acid template comprising a target nucleic acid and a universal priming site to a plurality of linked capture probes comprising a target probe having affinity to at least a portion of the target nucleic acid sequence, the target probe linked to a universal primer, wherein the exposing step occurs under conditions that require binding of the target probe to the target nucleic acid sequence to permit binding of the universal primer to the universal priming site;
extending the universal primer using rolling circle amplification.

21. The method of claim 20, wherein the circular nucleic acid templates are located in situ in a cell.

22. The method of claim 21, further comprising identifying the nucleic acid sequence and location within the cell using spatial sequencing analysis.
